(19) United States
(12) Patent Application Publication
(10) Pub. No.: US 2006/0122548 A1
(43) Pub. Date: Jun. 8, 2006

(54) Devices and methods of treatment of wounds and burns and related impaired blood circulation problems

(76) Inventor: Leonard Abrams, King of Prussia, PA (US)

Correspondence Address: Todd A. Norton;REED SMITH LLP, 2500 One Liberty Place, 1650 Market Street, Philadelphia, PA, 19103-7301, US

(21) Appl. No.: 11/093,267

(22) Filed: Mar. 29, 2005

Related U.S. Application Data
(60) Provisional application No. 60/634,067, filed on Dec. 6, 2004.

(51) Int. Cl. A61F 15/00; A61F 13/00

(52) U.S. Cl. 602/48; 602/41; 602/42; 602/46

(57) ABSTRACT

A device for treating wounds on an individual is disclosed. The device includes a flexible foam, a treatment suitable for treating the wound placed in relative proximity to the foam, and a flexible fabric with recoverable properties substantially circumferencing the wound and the treated foam. The flexible fabric maintains the treated foam in substantial proximity to the wound to allow the treatment to treat the wound. A bandage suitable for treating wounds on an exterior limb is disclosed. The bandage includes a butterfly shaped flexible fabric, at least one Hook and loop material type fastener attachment located on the edge of the butterfly shaped flexible fabric, at least two foam layers located axially in the center of the butterfly shaped flexible fabric, and a medication suitable for treating the wound proximately located with respect to at least a first of the at least two foam layers. The butterfly shaped flexible fabric is suitable to be wrapped around the limb and fastened using the at least one Hook and loop material type fastener attachment, and the wrapped fabric maintains the at least two foam layers in proximate location with the wound such that the medication is located appropriately with the wound to provide treatment.

### FIELD OF THE INVENTION

The present invention is a device designed for the treatment of wounds and bums, through the enhancement of circulation while providing an active anti-microbial agent to deter the risk of infection.

### BACKGROUND OF THE INVENTION

When the body sustains a wound or a bum, the trauma to the tissue is susceptible to infection and possible necrosis. Commonly, most wounds, pending on the severity, heal easily with little or no significant treatment. Some wounds, however, pending on the patient, require more treatment in order to assure proper healing.

For many patients, either due to their failure to seek care altogether or get proper medical treatment or because of the body's own inability to ward off infection results in wound or a burn that becomes severely infected. Contributing many times to the body's slow process of healing is the lack of presence of adequate circulation to the site of injury. Such circumstances require the need for a device that will provide enhanced circulation to the site while providing a zone of inhibition from infection.

Therefore, the need exists for a treatment device and method for the treatment of wounds and bums, and additional for a treatment device and method to treat wounds and bums in patients suffering from circulatory problems.

### BRIEF SUMMARY OF THE INVENTION

A device for treating wounds on an individual is disclosed. The device includes a flexible foam, a treatment suitable for treating the wound placed within or in relative proximity to the foam, and a flexible fabric with recoverable properties substantially circumferencing the wound and the treated foam. The flexible fabric maintains the treated foam in substantial proximity to the wound to allow the treatment to treat the wound.

A bandage suitable for treating wounds on an exterior limb is disclosed. The bandage includes a butterfly shaped flexible fabric, at least one Hook and loop material type fastener attachment located on the edge of the butterfly shaped flexible fabric, at least two foam layers located axially in the center of the butterfly shaped flexible fabric, and a medication suitable for treating the wound proximately located with respect to at least a first of the at least two foam layers. The butterfly shaped flexible fabric is suitable to be wrapped around the limb and fastened using the at least one Hook and loop material type fastener attachment, and the wrapped fabric maintains the at least two foam layers in proximate location with the wound such that the medication is located appropriately with the wound to provide treatment.

### BRIEF DESCRIPTION OF THE SEVERAL VIEWS OF THE DRAWINGS

Understanding of the present invention will be facilitated by consideration of the following detailed description of the preferred embodiments of the present invention taken in conjunction with the accompanying drawings, in which like numerals refer to like parts and in which:

FIG. 1 depicts a plan view of a first bandage according to the present invention;

FIG. 2 depicts a side elevation of the bandage of FIG. 1;

FIG. 3 depicts a side elevation of a second bandage embodiment having a second, different edge profile;

FIG. 4 depicts a side elevation of a third bandage embodiment having yet a third, different edge profile;

FIG. 5 depicts a top plan view of a fourth bandage embodiment different in size from the earlier embodiments;

FIGS. 6A and 6B illustrate a bandage embodiment of the present invention;

FIG. 7 illustrates one of the bandages of the previous figures being used on a patient's limb with an auxiliary foam pad;

FIG. 8 depicts a side elevation of an auxiliary foam boot which may be used with or without a bandage of the present invention;

FIG. 9 depicts a side elevation of an auxiliary foam low boot, which may be used with or without bandages of the present invention;

FIG. 10 depicts a top plan view of the boots of FIGS. 8 and 9;

FIG. 11 depicts a leg bandage according to an aspect of the present invention;

FIG. 12 depicts a bandage according to an aspect of the present invention; and,

FIG. 13 depicts an embodiment of the present invention including a foam according to an aspect of the present invention.

### DETAILED DESCRIPTION OF THE INVENTION

It is to be understood that the figures and descriptions of the present invention have been simplified to illustrate elements that are relevant for a clear understanding of the present invention, while eliminating, for the purpose of clarity, many other elements found in typical treatment devices, methods and systems. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

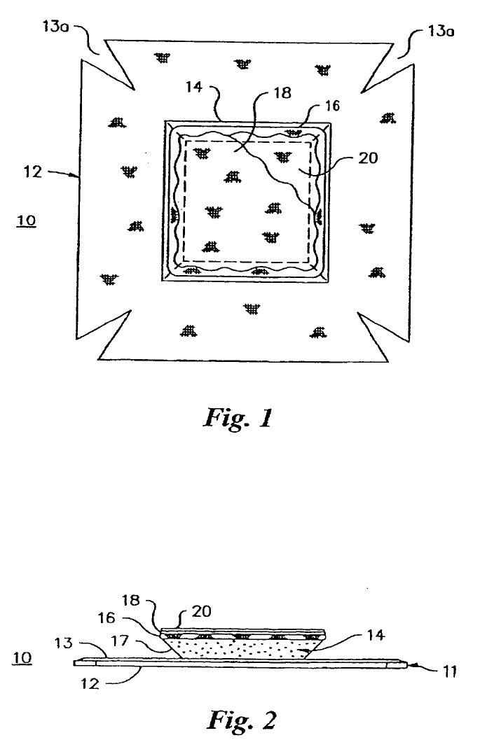

FIGS. 1 and 2 depict an exemplary bandage embodiment according to the present invention. Bandage 10 may include an outer, flexible adhesive member 11, in the form of a flexible strip 12, with a layer of adhesive 13, such as an FDA approved medical grade adhesive, on an inner major planar side. An outer major planar side of strip 12 forms an outer side of bandage 10. Bandage 10 further includes a flexible foam inner member 14 that may be at least generally planar and that may be generally centered on strip 12. An adhesive, or other adhering agent, such as Hook and loop material type fastener, for example, on inner major side of the strip may be used to fix one major side, such as a proximal side of inner member 14 facing strip 12, to strip 12, and at least some of adhering agent 13 may be exposed or exposable under a release strip (not depicted) on flexible strip 12 extending around the foam inner member 14, thereby allowing for the securing of bandage 10. A thin liner 16 formed by a layer of conventional bandage, such as gauze or other similar material, conventionally used to directly contact an open wound may be provided on the remaining, distal major side of foam member 14 facing away from flexible strip 12, so as to prevent direct contact of the foam with the user's skin or wound. The foam may be flexible, and may be, for example, a polymer-based foam of a density selected to respond to arterial and/or veinal circulatory system pulses, such as by allowing for deformation, by the pulses, and a springing back, by the foam, to its original position after the arterial or veinal blood vessel begins to relax. Thus, the foam may act like an auxiliary, external blood pump. Alternatively, minimal contact of the foam with the body may be employed. In such a configuration, a contra-compression may be used, to thereby provide only slight contact to the wound or burn.

The foam member may be open celled for air passage therethrough. One such foam may be a polyether foam grade, which may have the following characteristics: an apparent density of between about 0.77 and 0.97 lbs/ft³; an indentation force deflection of about 28-34 lbs to achieve a 25% deflection in a four (4) inch thick piece of foam, fourteen (14) inches square in size; a tensile strength of about 10 PSI (KPA); an ultimate elongation of 100%; a tear strength of about 1 lb per inch; and a compression set of about 10% loss when compressed 90% for about 22 hours at 70° C. Similar foams in a range of densities of at least between 0.5 and 6 lbs/ft³ may likewise be used successfully with the present invention.

The foam member, or one or more layers thereof, may be about one-half to about one and one-half inches thick. The thinner material may be suggested for grade 1 dermatological conditions (redness) while a thicker material may be suggested for all dermatological conditions above grade 1. Foams thicker than about one and one-half inch might also be used. Forms of foam other than sheet, and other porous or otherwise gas filled flexible solid materials, might be used in place of conventional sheet foam described above. Foams may be placed in multiple layers to obtain maximum therapeutic effect, and the characteristics of each layer may differ. For example, the thickness of each layer may be varied, and the medications or treatments at each layer may be varied, such as to allow timed release of different medications or treatments, at different times, by different layers. Further, for example, certain treatments may be undesirable for placement in direct contact with skin, but may nonetheless be therapeutic in nature if placed proximate to the afflicted area. The layers of foam may number two, three, or four, for example.

Treatments placed in or on the foam may take the form of a liquid, such as a salve or the like, or the form of a crystal, such as a nano crystal, for example. The treatments may be or include therapeutic remedies to speed the curing of the patient's affliction. Such treatments may include analgesics, pain relievers, antiseptics configured to clean contaminated wounds and bums, antibiotics for infections or sepsis, medicated dressings, corticosteroid hormones, tetanus shots, growth factors or other substances that stimulate healing. Treatments may further include characteristics that match, or simplify association with, the characteristics of the foam used in the bandage. For example, foam may contain any anti-microbial agents which will ensure a decrease in patient's bioload to a wound thus preventing the risk of infection. Examples may include silver in various forms, or other anti-biotic therapy. For example, AlphaSan RC2000 powder from Milliken contains 10% silver. This may be a high grade and high concentration of silver in a crystallized form, which may allow for heavier loading of the foam with the crystals than lower grade silver content powders. For example, the silver sodium hydrogen zirconium phosphate may include a silver ion concentration of about 0.5 to about 1.0%. Further, the silver sodium hydrogen zirconium phosphate may include a silver ion concentration of about 0.3 to about 1.5%. Further, for example, this silver may be provided in the form of a nano crystallized silver sodium hydrogen zirconium phosphate, which based on the loading of the foam, may provide a silver ion concentration of 8.5%×0.10 (10%)=0.85%.

Liner 16 may be any conventional cotton, polymer or cotton/polymer blend bandage gauze from any of a variety of suppliers. Liner 16 may be as thin as practicable. The gauze may be treated with a coating such as Teflon, or may be made from such a material to prevent sticking to the wound. For other materials, a coating 18 may be provided on the gauze member 16 of A+D Ointment or other suitable, dermatological ointment. A layer 20 of medication, e.g., an antibiotic and/or antifungal ointment, may be applied on the exposed upper surface over or in place of the dermatological ointment for direct contact with the afflicted area.

Liner 16 and underlying foam member 14 may be of a size to fully span and extend beyond the edges of any afflicted area, the wound or burn, and adhering member 12 may extend at least two more inches beyond the outer periphery of foam member 14 (or foam member 14 with gauze 16) on all sides. Alternatively, the foam member or members may extend beyond the outer periphery of the adhering member, as discussed further hereinbelow.

As illustrated in FIG. 2, any sharply delineated edges or other potential contact sources on bandage 10 may be eliminated. To that end, thinnest liner 16 may be used to minimize any step around the outer perimeter of liner 16. Also, the foam material, which may be supplied in generally planar sheets, may be provided with side edges 17 which extend transversely to the major sides of foam inner member 14 and flexible strip 11 and which may be other than perpendicular to the generally planar and parallel opposing major sides of foam member 14.

More specifically, each foam member 14 has an outer perimeter formed by side edges 17, which extends around and between its pair of opposing major sides. The foam inner member may diminish in thickness or change in shape in the outer perimeter around the foam inner member sufficiently smoothly to avoid creation of any abrupt contact change along the skin of the individual wearing the bandage. Such an abrupt contact change may hinder blood flow through the skin under the foam inner member. Thus, the perimeter may lack any substantial discontinuity (e.g. transversely extending step or other change in height of the member or the like along the periphery) sufficiently abrupt to create a discontinuous contact change along the skin of the individual receiving the bandage along the periphery of the foam inner member. Generally speaking, a discontinuity may be caused by a sharp edge, that is, a side edge with a surface perpendicular or at least sufficiently near perpendicular to the plane of foam member 14 to cause the skin at the perimeter of the foam inner member to fold around the edge of the perimeter sufficiently severely to reduce or stop the flow of blood through the fold of the skin.

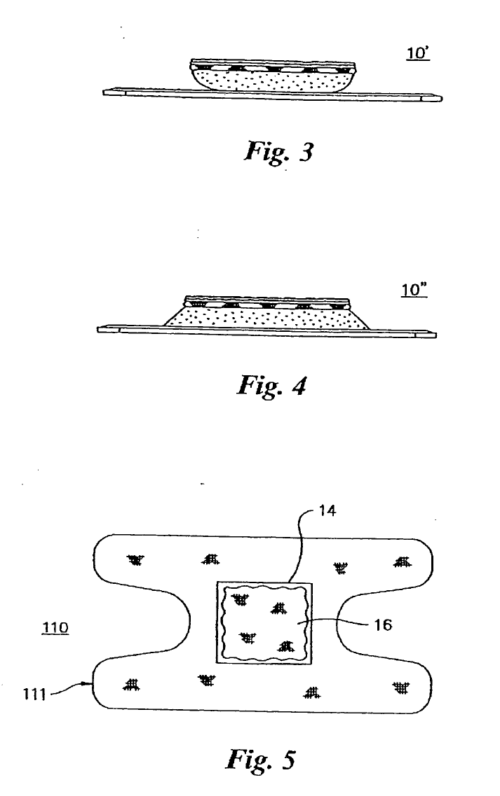

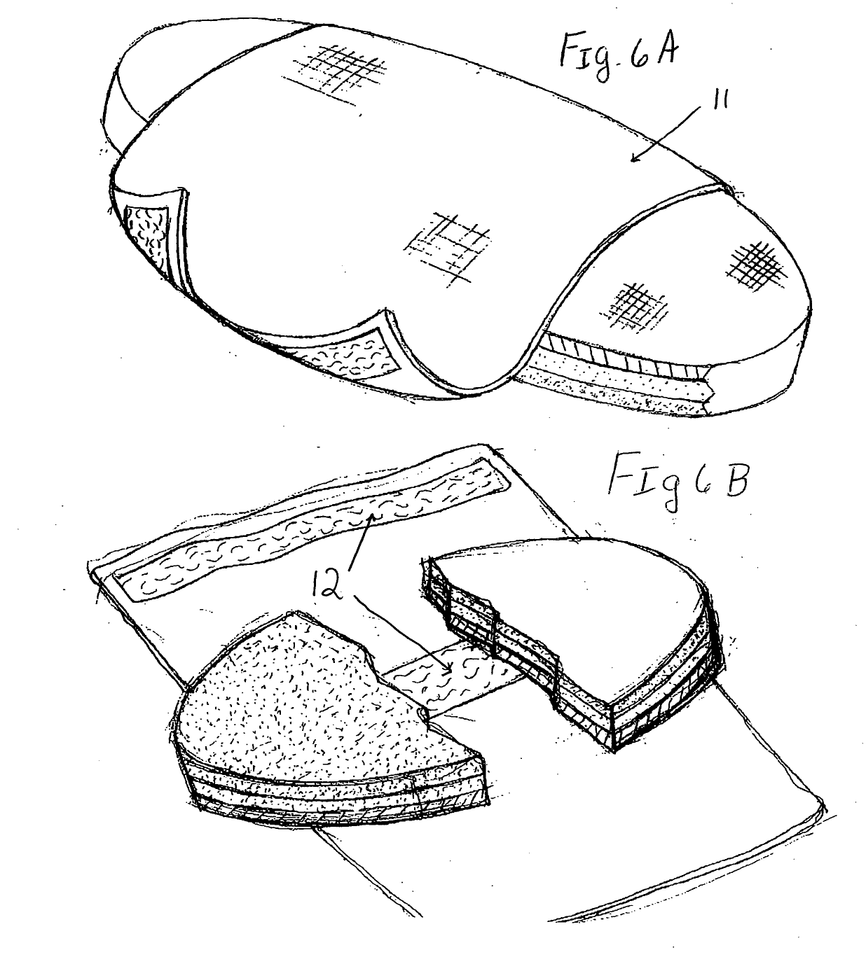

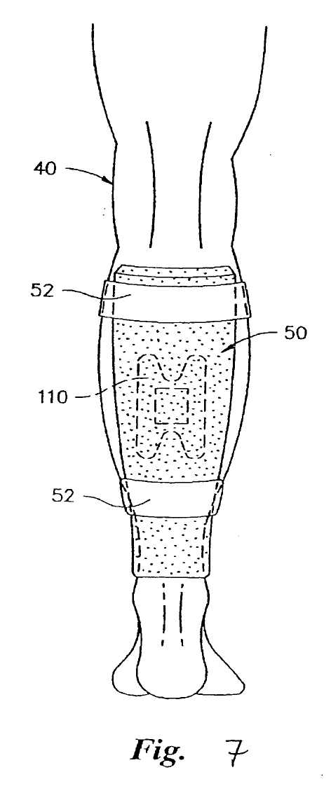

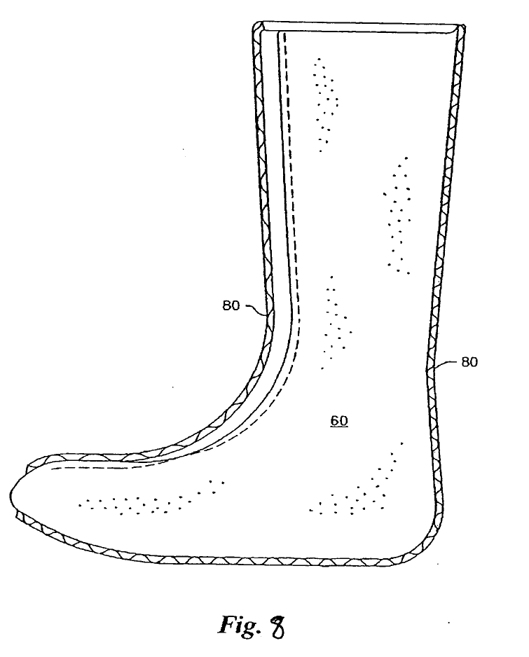

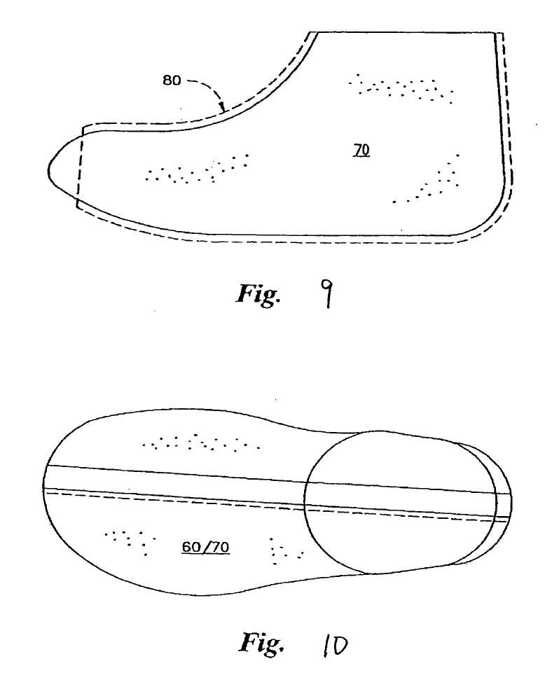

Bandage 10 may also take the form of a tapered edge surface which may need not be straight. As may be evident to those possessing an ordinary skill in the pertinent arts, undercuts such as those depicted in FIGS. 2 and 3 may provide at least as uniform a contact application as the tapered cut of FIG. 4, while providing more uniform contact of the foam to the wearer's skin over the entire area of the foam. Unless such edge treatment is provided to the foam, or some other treatment is provided to equivalently eliminate any abrupt transition along the outer periphery of foam member 14, a discontinuous contact change may be created near the afflicted area, and such a transition may interfere with blood flow through and immediately below the skin to the afflicted area. To the same end, adhering member 11 may be as thin as practicable to eliminate the creation of a relatively sharp edge at the outer periphery of either member, because such edge may also act as a contact source cutting off the blood flow to the skin, such as especially where the bandaged area of the wearer must be placed upon a support surface.

Bandage 10 may be applied to the afflicted area with or without medication or other such materials on its exposed treatment surface and adhered to the body of the patient for a period of time depending upon a treatment selected. Bandages of the present invention may be applied to the afflicted area with moderate contact, something between what would be regarded as a loose fitting and a tight fitting for a bandage. The bandage may be applied to the afflicted area with enough contact so that circulatory system pulses are transmitted to the foam and the foam may be able to compress and relax in response to the circulatory system pulses but not so tight as to curtail or diminish the occurrence or strength of the circulatory pulses. Obviously, the optimal contact will vary for each case and may depend on the treatment delivery requirements. The bandage may be removed after a period of time, such as three days, for example, and the afflicted area may be cleaned and treated and a new bandage applied, if necessary.

Bandages of the present invention may be provided in various sizes and shapes. The proportions of adhering member 11 may be varied with respect to flexible foam member 14 and/or liner 16, as indicated by bandage embodiment 110 shown in FIG. 5. FIG. 5 illustrates bandage 110 with another form of cut-outs, such as a butterfly. It will be apparent to those possessing an ordinary skill in the pertinent arts that other shapes may be used and needed for the treatment of wounds. The present invention may be adapted to be used with virtually any conventional or special form of adhesive bandage.

While generally square/rectangular bandages and components have been shown, these are intended to only be illustrative. Showing such shaped bandages is based, in large part, upon the widespread availability of rectangularly shaped bandages and bandage components and requirements of the present invention. Adhesive bandages 10, 110 as described above, may be used on virtually any exposed surface of the human body which may become wounded or burned.

FIGS. 6A and 6B illustrate an embodiment of a bandage of the present invention employing smooth outer edges of foam, lacking discontinuity, wherein the foam member may have multiple layers, and wherein the foam member layers may extend beyond the outer periphery of adhesive member 11. A back view and a breakaway view of a three layered foam member are illustratively shown, and the base layer of the foam member may be adhered to the bandage. Adhered point or points 12 whereat the foam may be adhered by an adhesive such as a glue, a hook and loop material type fastener, or other sanitary and medically safe adhering mechanism. Further, the adhered point may be a removable adhesive wherein the foam may be removably attached to the bandage. There may be one or more adhered points, and foam adhesion 12 that makes up the adhered points may be, for example, one or more points, one or more strips, or any other pattern of adhesive.

FIG. 7 illustrates schematically the use of one of bandages 110 of the present invention on a limb, for example, a leg 40, to increase the flow of blood into the afflicted area from beyond bandage 10 itself. A separate strip or patch 50 of the flexible foam material may be applied over bandage 10 or 110, overlapping the bandage and extending beyond the bandage, such as in a longitudinal orientation with respect to the limb, to improve circulation along the limb through of the afflicted area. Strip or patch 50 may be held in position against limb 40 by one or more strips 52 of material, such as the same adhesive cloth material mentioned above for use as adhering member 11. Strip 50 may define a corridor along which the flow of blood may be assisted or boosted. A length of fabric tubing with recoverable properties may be applied over the affected limb and over strip 50 to hold substantially, if not all, of patch 50 against limb 40 to thereby provide greater foam/skin contact and more efficient auxiliary pumping action by strip 50. FIG. 7 illustrates use of foam strip 50 to improve circulation in the calf and below. Strip 50, for example, may extend, in such an embodiment, from just above the tendon-Achilles insertion of the tendon to the top of the calf muscle just below the knee. While two adhesive strips 52 have been depicted, it would be evident to a person having ordinary skill in the pertinent arts that a greater number of strips may be used. Alternatively, the foam strip 50 may be held in place with a cloth wrap or a length of fabric tubing with recoverable properties, such as a stocking.

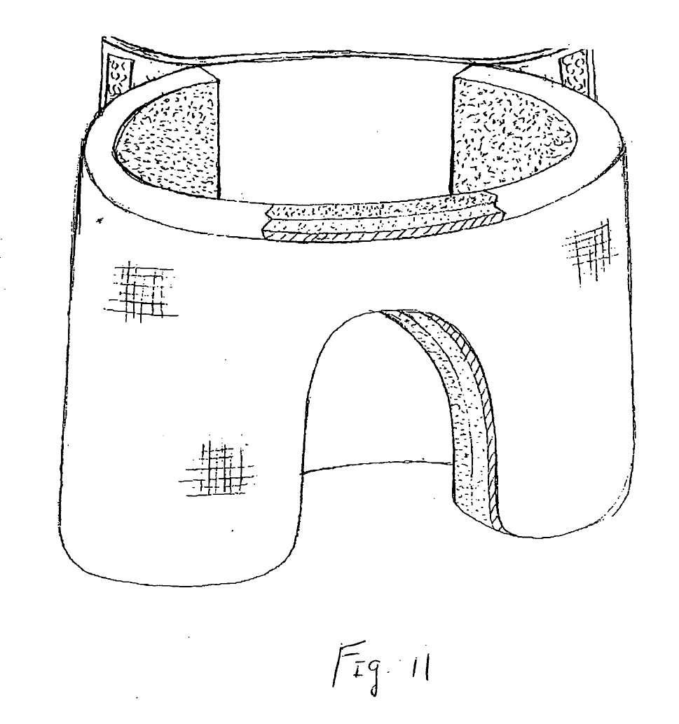

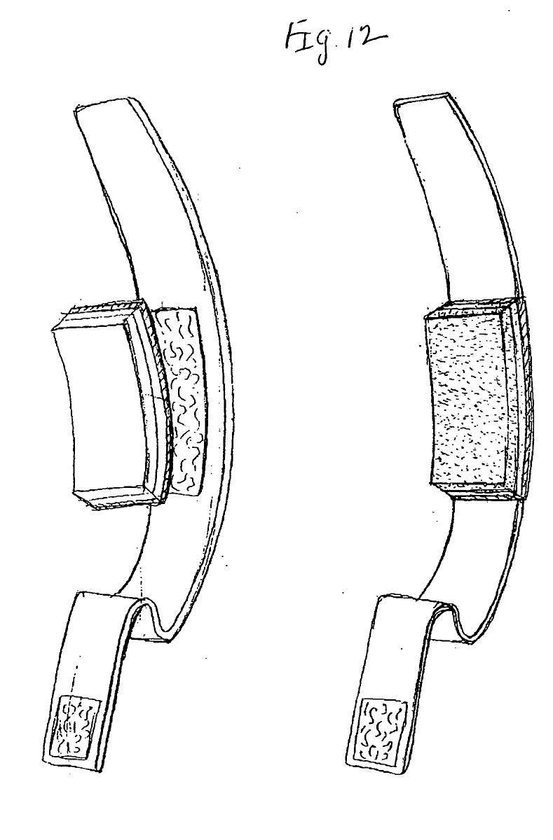

Feet and lower legs are typically the extremities initially or most seriously affected by poor blood circulation. To that end, FIGS. 8-10 depict an exemplary high foam boot 60 and a low foam boot 70, respectively, which may be used in place of a conventional length of foam-like strip 50 on the foot or foot and lower leg area. Boots 60, 70 may be formed from the aforesaid foam material, such as one and one-half inch thick foam material, and applied over the user's foot and over any bandage 10 or 110 applied directly to an affected area. Boots 60, 70 may be held in place in close contact with the skin of the foot or foot and lower leg by suitable means such as an fabric tubing with recoverable properties, such as a bandage or a length of the fabric tubing 80 of the type referred to above. Boots 60,70 may be made manually by wrapping a length of flexible foam sheet around the foot or foot and lower leg and cutting away the overlapping portion of the foam to leave a single, foam layer covering. This covering may be held in place by fabric tubing with recoverable properties, such as a bandage or fabric tubing. It may also be apparent to those possessing an ordinary skill in the pertinent arts that such boots may be specially made, such as in various sizes, and may be provided with such amenities as Hook and loop material type fastener closures for long term durability and ease of use. The high boot may be designed to extend upward to the tuberosity of the tibia at the knee joint. It may be also envisioned that such boots may be made with a foam having its own fabric tubing with recoverable properties character or may be formed with an fabric tubing with recoverable propertiesized material so that it may be slipped on and off and worn like a slipper-sock. Boots 60,70 may be used without underlying bandages to encourage foot or foot and lower leg blood circulation even before circulatory injuries arise.

FIG. 11 depicts an exemplary embodiment of a leg bandage, wherein the foam makes up at least a portion of the outer portion of the bandage, and wherein a remaining portion of the bandage may be made up of adhering member 11. In such an embodiment, the adhering layers may contact the bandage to the afflicted area and may be placed on opposing ends of adhesive member 11, such that the opposing ends of the adhesive member may be adhered to the foam member. A resizable bandage, such as for placement around a limb, may be provided. In this configuration, a protective layer, or coating, may be provided on the outer periphery of the foam member.

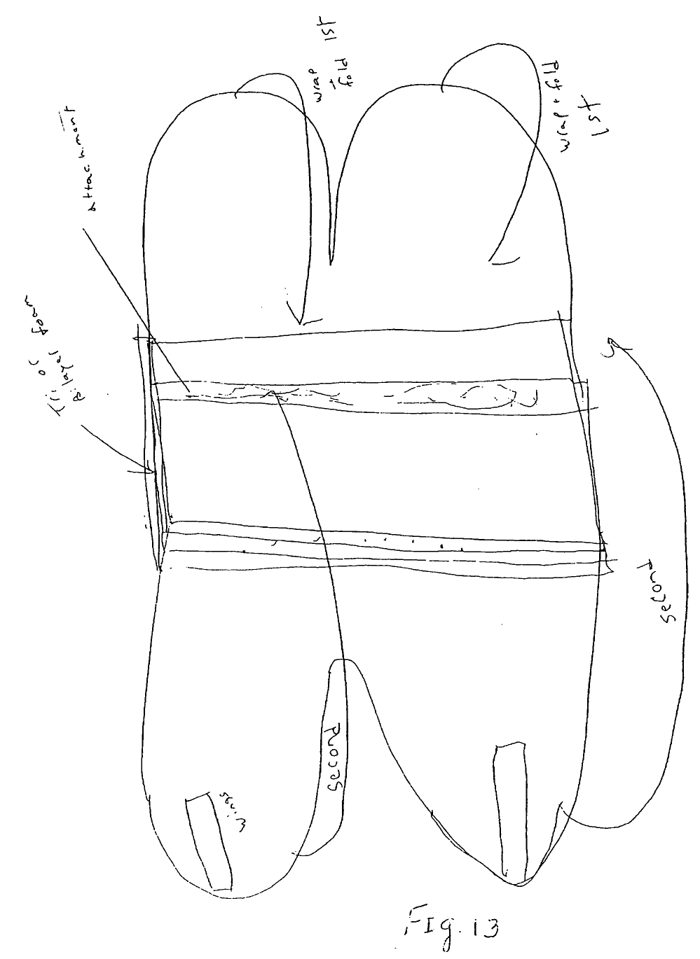

FIG. 12 depicts an embodiment of a bandage of the present invention wherein a multi-layered foam may be removably provided in a smaller bandage that provides more convenient range of motion and better breathability. As shown in FIG. 12, the adhering member may be a strip, which may be of a suitable length and width to cover afflicted areas of various sizes, while wrapping around, for example, an afflicted limb. The strip may be provided, for example, in a variety of sizes. The foam illustratively shown may be permanently or removably secured to the strip, and may be a multiple layered foam. The strip may include one or more adhering layers 13, such as a single adhering layer on one end that seals to the outer face of an opposing end of adhering member 11 strip, or such as an adhering layer on one end that seals to adhering layer 13 at the opposing outer face of adhering member 11. According to an aspect of the present invention including multiple adhering layers 13, the adhering layers may provide a permanent or a removable adhesion, such as a medically safe glue, epoxy, Hook and loop material type fastener, or grabbing tab, for example.

FIG. 13 depicts an embodiment of the present invention including a bi or tri layer foam that may be removably attached to a multi-point adhesive, adjustable fit bandage. The bandage illustratively shown may include two or more “wings”, wherein each wing includes, on the inner part of the adhesive member, an adhesive, such as a Hook and loop material type fastener, and wherein the outer part of each opposing wing likewise includes an adhesive acceptor, such as a Hook and loop material type fastener acceptor. The adhesive on the outer part of the opposing wing may be of a length sufficient to allow the attachment of the inner part of the first wings at various locations along the outer part of the opposing wings, thereby providing a bandage that may be varied in size, and which may be further varied in sizes at different points within the bandage itself dependently upon the number of wings provided. The foam may be removably attached to allow changing of the foam without changing of the bandage, such as to allow for placement of foams of different numbers of layers or different types of medication onto the bandage.

Those of ordinary skill in the art will recognize that many modifications and variations of the present invention may be implemented without departing from the spirit or scope of the invention. Thus, it is intended that the present invention cover the modification and variations of this invention provided they come within the scope of the appended claims and their equivalents.

1. A device for treating a wound on an individual, said device comprising:
a flexible foam;
at least one treatment placed at least partially within said foam, said at least one treatment suitable for treating the wound, wherein said flexible foam at least partially releases said at least one treatment within substantial proximity to the wound over time; and,
a flexible fabric with recoverable properties substantially circumferencing the wound and said treated foam,
wherein said flexible fabric maintains said treated foam in substantial proximity to the wound to allow said at least one treatment to treat the wound.

2. The device of claim 1, wherein said flexible foam is polymer based.

3. The device of claim 1, wherein said flexible foam is open celled.

4. The device of claim 3, wherein said open celled foam is suitable for allowing airflow to the wound.

5. The device of claim 1, wherein said flexible foam has a thickness in the range of 0.5 to 1.5 inches.

6. The device of claim 1, wherein said device does not substantially hinder the range of motion of the individual.

7. The device of claim 1, wherein said device does not substantially hinder blood flow to the wound.

8. The device of claim 1, wherein said device does not substantially limit circulation of the individual.

9. The device of claim 1, wherein said at least one treatment includes silver sodium hydrogen zirconium phosphate.

10. The device of claim 9, wherein said silver sodium hydrogen zirconium phosphate of said at least one treatment includes a silver ion concentration of about 0.85%.

11. The device of claim 9, wherein said silver sodium hydrogen zirconium phosphate of said at least one treatment includes a silver ion concentration of about 0.5 to about 1.0%.

12. The device of claim 9, wherein said silver sodium hydrogen zirconium phosphate of said at least one treatment includes a silver ion concentration of about 0.3 to about 1.5%.

13. The device of claim 9, wherein said silver sodium hydrogen zirconium phosphate of said at least one treatment is in the form of a nanocrystal.

14. The device of claim 1, wherein said device is applied with substantially minimal contact to the wound area.

15. The device of claim 1, wherein said device is applied using contracompression.

16. The device of claim 1, further comprising a liner placed substantially adjacent to said foam distal to said flexible fabric.

17. The device of claim 16, wherein said liner is coated so as to prevent sticking to said wound.

18. The device of claim 17, wherein said coating is a fluoropolymer.

19-29. (canceled)

30. The device of claim 1, wherein said flexible foam is multi-layered.

31. The device of claim 30, wherein at least two layers of said multi-layered foam have different thicknesses.

32. The device of claim 30, wherein at least two layers of said multi-layered foam comprise different foam characteristics.

33. The device of claim 30, wherein individual ones of said at least one treatment are each placed at least partially within different selected layers of said multi-layered foam.
